(19) United States
(12) Patent Application Publication
(10) Pub. No.: US 2024/0050740 A1
(43) Pub. Date: Feb. 15, 2024

(54) ACTIVE IMPLANTABLE MEDICAL DEVICE COMPRISING APPLICATION SPECIFIC INTEGRATED CIRCUIT AND METHOD THEREOF FOR HEATING CONTROL

(71) Applicant: Norbert Kaula, Arvada, CO (US)

(72) Inventor: Norbert Kaula, Arvada, CO (US)

(21) Appl. No.: 18/493,778

(22) Filed: Oct. 24, 2023

Related U.S. Application Data
(63) Continuation of application No. 18/295,234, filed on Apr. 3, 2023.
Application No. 18/295,234 is a continuation of application No. 17/933,108, filed on Sep. 18, 2022, now abandoned.
Application No. 17/933,108 is a continuation of application No. 17/001,584, filed on Aug. 24, 2020, now abandoned.
(60) Provisional application No. 62/890,571, filed on Aug. 22, 2019.

(51) Int. Cl. A61N 1/08

(52) U.S. Cl. CPC A61N 1/086

(57) ABSTRACT

The present invention provides an active implantable medical device (AIMD) comprising an implantable lead, an application specific integrated circuit (ASIC) within a hermetic enclosure of the AIMD, and a sensing and cancellation wave output from the ASIC supplying to the lead. The invention also provides a method of reducing heating of an AIMD and a lead thereof.

### CROSS-REFERENCE TO RELATED APPLICATIONS

This application expressly claims the benefits and priority based on U.S. Provisional Application No. 62/890,571 filed Aug. 22, 2019, which is incorporated herein by references as if set forth in its entirety.

### FIELD OF THE INVENTION

The present invention generally relates to an active implantable medical device (AIMD) comprising an application specific integrated circuit (ASIC) and method thereof for heating reduction. Some embodiments provide a medical device comprising an ASIC for detecting and cancelling off at least a part of negative effect of an external magnetic/electrical field, a device and a system thereof, and a method thereof. Although the invention will be illustrated, explained and exemplified with an ASIC that detects the radiofrequency during a MRI exam when a patient enters a MRI, shuts down the IPG safely, and generates and outputs a cancellation voltage to reduce heating of the lead and IPG, it should be appreciated that the present invention can also be applied otherwise and/or in other fields.

### BACKGROUND OF THE INVENTION

A harmful interaction of an object with MR equipment, into which the object is brought into, especially with the static magnetic field, the gradient fields and the RF fields of the MR equipment, can pose hazards to patients or other persons. Interactions of medical devices with the MR environment may result in serious injuries and even death of patients. Potential direct causes of hazards include direct causes such as (1) mechanical causes, including magnetically induced displacement force, torque, and vibration, (2) electromagnetic causes, including induction (heating, stimulation) and discharge (spark gap), and (3) acoustic causes. Potential indirect causes of hazards include malfunction of vital components such as valves, monitors and pumps. Other possible safety issues to consider for the hazard assessment include, but are not limited to, thermal injury, induced currents/voltages, interaction with the switched gradient field (dB/dt) for all items that may go inside the magnet bore, electromagnetic compatibility, neurostimulation, acoustic noise, interaction among devices, and the malfunction of the item and the malfunction of the MR equipment and accessories.

For example, during magnetic resonance imaging (MRI) procedures, for patients with active implantable medical devices (AIMDs), the AIMDs can interact with radio-frequency (RF) fields produced by the MRI RF coil. Such interactions result in the induced voltage/current on device circuits which can cause device malfunction or damage and the induced heating in human tissues which can cause tissue injury. Thus, the MRI RF safety for AIMD is important for patients.

Advantageously, the present invention provides a novel active implantable medical device (AIMD) comprising an application-specific integrated circuit (ASIC) and method thereof for heating reduction.

### SUMMARY OF THE INVENTION

One aspect of the present invention provides an active implantable medical device (AIMD) comprising an implantable lead, and an application specific integrated circuit (ASIC) within a hermetic enclosure of the AIMD, wherein the ASIC senses an external signal and generates a cancellation wave output and supplies it to the lead to reduce its heating; and/or the ASIC senses an external signal and adaptively adjusts an impedance of a non-lead portion of the AIMD to minimize an induced heating of the lead.

Another aspect of the invention provides a method of reducing heating of an active implantable medical device (AIMD) and a lead thereof. The method includes:
(i) providing an application-specific integrated circuit (ASIC) within a hermetic enclosure of the AIMD;
(ii) sensing an external signal by the ASIC; and
(iii) generating a cancellation wave output and supplying it to the lead to reduce its heating; and/or adaptively adjusting an impedance of a non-lead portion of the AIMD to minimize an induced heating of the lead.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 demonstrates an active implantable medical device (AIMD) in accordance with an exemplary embodiment of the present invention.

FIG. 2 is a flow chart of the method of reducing heating of an AIMD and a lead thereof in accordance with an exemplary embodiment of the present invention.

FIG. 3 illustrates a patient with an AIMD and lead(s) inside a MRI apparatus in accordance with an exemplary embodiment of the present invention.

FIG. 4 shows a specific method of reducing heating of an AIMD and a lead thereof in accordance with an exemplary embodiment of the present invention.

FIG. 5 shows detected RF from MRI, and cancellation wave phase shifted by 7C back into the lead in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only, and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

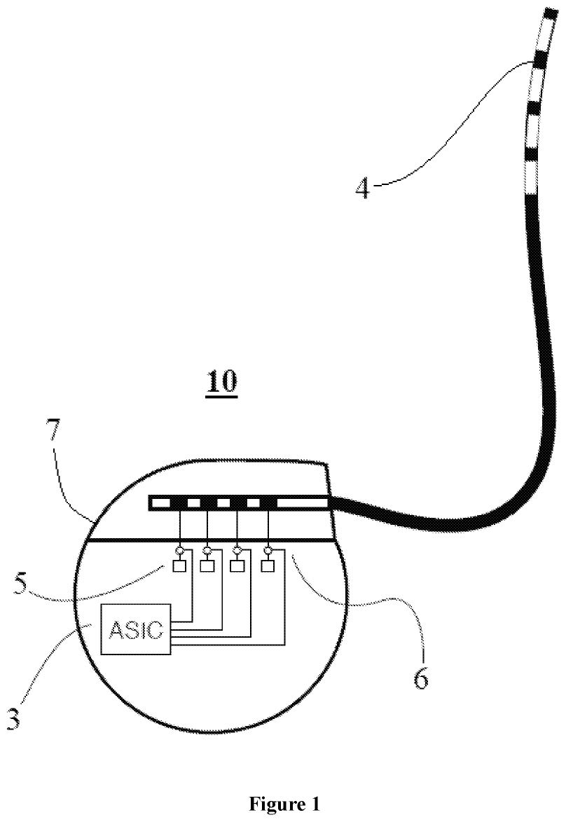

With reference to FIG. 1, an active implantable medical device (AIMD) 10 includes an implantable lead 4, an application-specific integrated circuit (ASIC) 3 within a hermetic enclosure 7 of the AIMD 10, and a sensing and cancellation wave output 6 from the ASIC 3 supplying to the lead 4. In preferred embodiment, the AIMD 10 is an implantable pulse generator (IPG). A stimulation output 5 may be generated in the AIMD 10 supplying to the lead 4. The ASIC senses (or detects, or measures) an external signal and generates a cancellation wave output and supplies it to the lead to reduce its heating. Alternatively or additionally, the ASIC senses (or detects, or measures) an external signal and adaptively adjusts an impedance of a non-lead portion of the AIMD to minimize an induced heating of the lead.

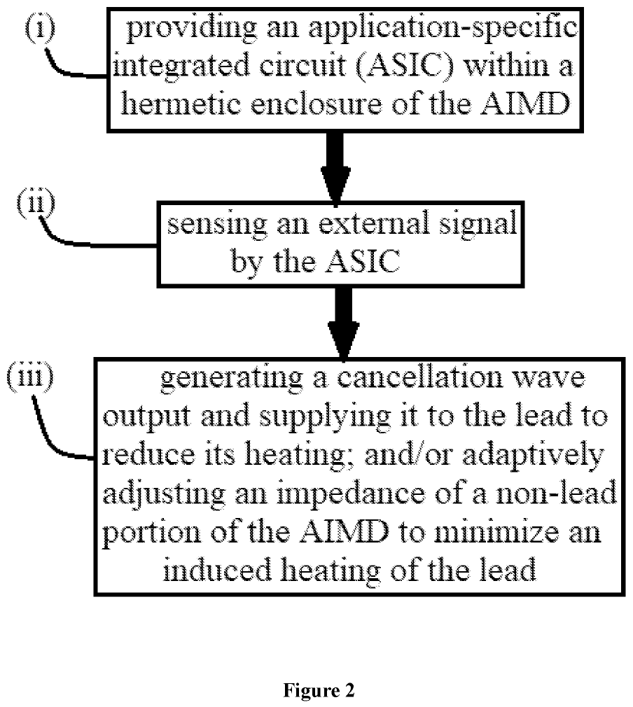

FIG. 2 is a flow chart of the method of reducing heating of an AIMD and a lead thereof. The method includes (i) providing an application-specific integrated circuit (ASIC) within a hermetic enclosure of the AIMD; (ii) sensing an external signal by the ASIC; and (iii) generating a cancellation wave output and supplying it to the lead to reduce its heating; and/or adaptively adjusting an impedance of a non-lead portion of the AIMD to minimize an induced heating of the lead.

In exemplary embodiments, the ASIC 3 may function as MRI safety sensing ASIC with active reverse voltage injection for the AIMD 10.

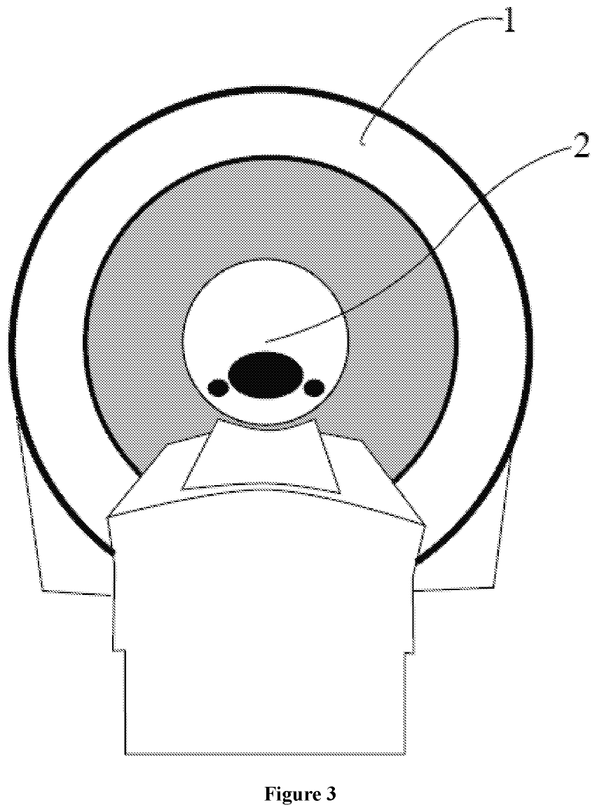

As shown in FIG. 3, a patient 2 with an AIMD and lead(s) are entering a MRI apparatus 1. Static magnetic Field and RF field can be 1.5 T—64 MHz or 3.0 T—128 MHz. The ASIC 3 can detect when a patient enters a MRI, shut down the IPG 10 safely, protect the circuitry, enter MRI mode, detect the MRI RF field, generate and output a cancellation voltage with the indent to reduce heating of the lead and the IPG.

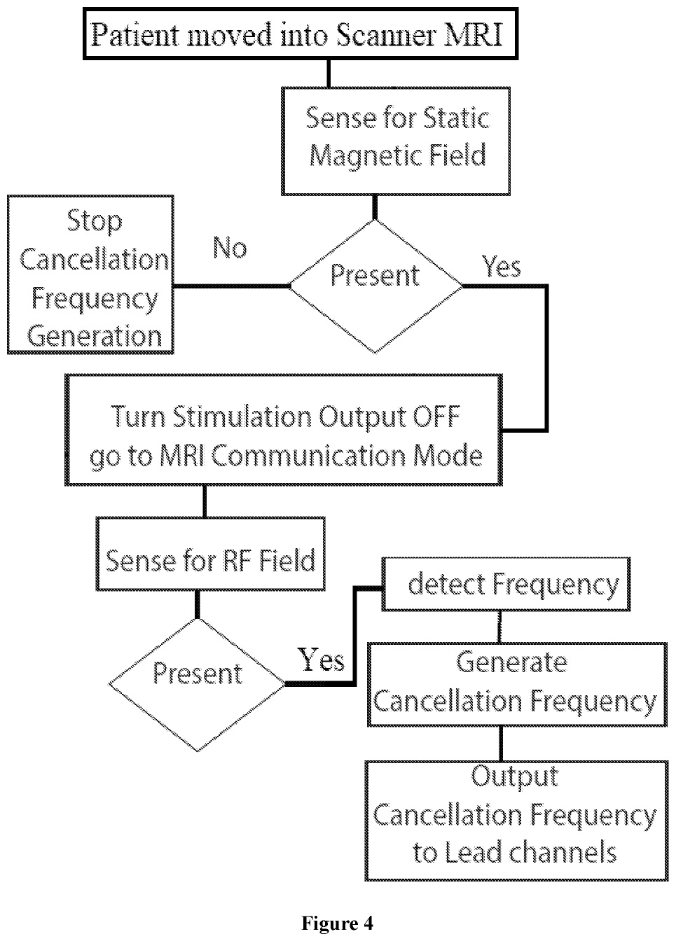

FIG. 4 shows a flow chart in which a patient is first moved into Scanner MRI; Sense for Static Magnetic Field (SMF); if SMF not present “No”, then Stop Cancellation Frequency Generation; if SMF present “Yes”, then Turn Stimulation Output OFF, go to MRI Communication Mode, and Sense for RF Field (RFF); if RFF present “Yes”, then detect Frequency, (1) Generate Cancellation Frequency, and Output Cancellation Frequency to Lead channels. Or (2) adjusts an impedance of a non-lead portion of the AIMD

In some other embodiments, the ASIC senses (or detects, or measures) an external signal and adaptively adjusts an impedance of a non-lead portion of the AIMD to minimize an induced heating of the lead. For example, the ASIC circuit may be used to (as step 1) detect the input RF signal induced on various electrodes during MR scanner, and to estimate the induced power at each electrode inside the ASIC. Based on the induced voltage level, the ASIC may be used to (as step 2) change the input impedance values between electrode and the ground. Go to step 1 to sense again. Keep on doing step 1 and step 2 until the maximum induced power is reached. Then, use the corresponding value for the rest of the MR imaging. By inducing the maximum power inside the IPG, the induced energy to the tip such as lead tip would be lower. Consequently, this will reduce the device tip heating.

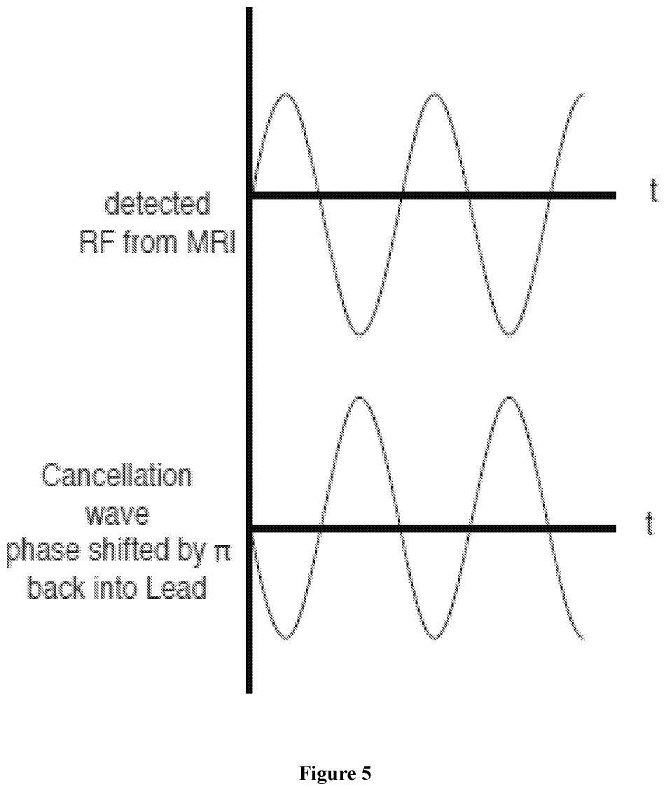

FIG. 5 shows detected RF from MRI, and cancellation wave phase shifted by 7C back into the lead.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. An active implantable medical device (AIMD) comprising an implantable lead, an application-specific integrated circuit (ASIC) within a hermetic enclosure of the AIMD, wherein the ASIC senses an external signal and generates a cancellation wave output and supplies it to the lead to reduce its heating; and/or the ASIC senses an external signal and adaptively adjusts an impedance of a non-lead portion of the AIMD to minimize an induced heating of the lead.

2. The active implantable medical device according to claim 1, which is an implantable pulse generator (IPG).

3. The active implantable medical device according to claim 1, further comprising a stimulation output 5 supplying to the lead 4.

4. A method of reducing heating of a lead of an active implantable medical device (AIMD), comprising (i) providing an application-specific integrated circuit (ASIC) within a hermetic enclosure of the AIMD; (ii) sensing an external signal by the ASIC; and (iii) generating a cancellation wave output and supplying it to the lead to reduce its heating; and/or adaptively adjusting an impedance of a non-lead portion of the AIMD to minimize an induced heating of the lead.
